(19) United States
(12) Patent Application Publication
(10) Pub. No.: US 2002/0015966 A1
(43) Pub. Date: Feb. 7, 2002

(54) Effector-specific protein assembly and uses thereof

(76) Inventors: David Dudley Moore, Bellaire, TX (US); Pavlos Pissios, Boston, MA (US)

Correspondence Address: CLARK & ELBING LLP, 176 FEDERAL STREET, BOSTON, MA, 02110-2214, US

(21) Appl. No.: 09/815,416

(22) Filed: Mar. 22, 2001

Related U.S. Application Data
(60) Provisional application No. 60/191,946, filed on Mar. 24, 2000.

(51) Int. Cl. G01N 33/53; G01N 33/542

(52) U.S. Cl. 435/7.9

(57) ABSTRACT

Disclosed herein are assays and kits for identifying effector molecules (such as ligands) based on their ability to mediate assembly of their cognate proteins (such as receptors).

### CROSS REFERENCE TO RELATED APPLICATIONS

This application claims priority from U.S. Provisional Application No. 60/191,946, filed Mar. 24, 2000.

### STATEMENT AS TO FEDERALLY SPONSORED RESEARCH

This invention was made in part with government support under NIH grant NIDDK 43382. The government therefore has certain rights in the invention.

### BACKGROUND OF THE INVENTION

In general, the invention relates to assays for effector molecules based on effector-specific assembly of proteins.

A large number of important biological events are controlled by the binding of an effector molecule to a protein. For example, many signal transduction events result from the interaction between either a protein kinase and its substrate or a receptor and its ligand. Due at least in part to the ubiquitous nature of these types of effector-mediated interactions, the design or isolation of pharmaceutical agents frequently targets such effectors or their protein partners.

Despite the utility of identifying effector molecules, however, current screening methods for effector molecules generally suffer from one or more shortcomings. For example, such assays frequently employ radioactive effectors as ligands, which can be difficult and expensive to produce, and inconvenient to use. In addition, many of these assays require complex, specialized equipment to measure effector binding. Moreover, technical limitations can prevent the application of many of these conventional assays to effectors that require relatively high concentrations or act with relatively low specificity.

### SUMMARY OF THE INVENTION

The current invention involves a novel means to detect effector specific assembly of proteins that is simple and broadly applicable, can use but does not require radioactive compounds, and is amenable to the identification of a wide range of potential effectors, including both small molecules and proteins.

In particular, the invention features a method for identifying an effector molecule which modulates the activity of a protein, the method involving: (a) providing at least two fragments of the protein; (b) contacting the fragments with a candidate molecule; and (c) assaying for assembly of the fragments, whereby a level of fragment assembly in the presence of the candidate molecule that is greater than the level in its absence identifies the candidate as an effector molecule which modulates the activity of the protein.

In preferred embodiments, the protein is a member of the nuclear receptor superfamily (for example, a thyroid hormone receptor, retinoic acid receptor, retinoid X receptor, or estrogen receptor), and one of the fragments includes the helix 1 domain of the nuclear receptor and the second of the fragments includes the helix 12 and ligand binding pocket of the nuclear receptor.

In other preferred embodiments of the assay, the effector molecule is a protein, ligand, corepressor, or antagonist; one or both of the fragments is provided by expressing a fragment-encoding nucleic acid; and the contacting step (b) occurs either in vivo or in vitro.

In one particular assay format, one of the fragments may be immobilized on a solid support, and the second of the fragments may be detectably labeled. In this format, the assaying step (c) involves detecting the label in association with the solid support.

In another assay format, the protein is a DNA binding protein, and the contacting step (b) occurs in the presence of a nucleic acid that includes the binding site for the DNA binding protein. In this format, the assaying step (c) involves detecting binding of the assembled fragments to the binding site.

In yet another assay format, one of the fragments is covalently bound to a DNA binding domain and the other fragment is covalently bound to a gene activation domain (and preferably at least one of the DNA binding or gene activation domains is a heterologous domain). In this assay format, the contacting step (b) is carried out in the presence of a reporter gene operably linked to a binding site for the DNA binding domain, and the assaying step (c) involves detecting expression of the reporter gene. This assay is preferably carried out in a yeast or mammalian cell.

In a related aspect, the invention features a kit for identifying a nuclear receptor effector molecule, the kit including: (a) a first fragment of the nuclear receptor, the first fragment including the helix 1 domain of the nuclear receptor; and (b) a second fragment of the nuclear receptor, the second fragment including the helix 12 and ligand binding pocket of the nuclear receptor.

In preferred embodiments, the protein is a member of the nuclear receptor superfamily (for example, a thyroid hormone receptor, retinoic acid receptor, retinoid X receptor, or estrogen receptor); and the effector molecule is a ligand, corepressor, or antagonist. At least one of the kit fragments may be detectably labeled and/or may be immobilized on a solid support. Alternatively, one of the fragments may be covalently bound to a DNA binding domain and the other fragment covalently bound to a gene activation domain (for example, a heterologous DNA binding domain or gene activation domain). In this case, the kit also includes a nucleic acid that includes a binding site for the DNA binding domain.

By “assembly” is meant the stable association of protein fragments and effector molecule(s). As used herein, this assembly must be of a nature that allows for the detection of the complex by either an in vivo or in vitro technique.

By “helix 1 domain” is meant that domain of a nuclear receptor that includes the first conserved helix of the nuclear hormone receptor ligand binding domain as defined by x-ray crystal structures, for example, those of the receptors for thyroid hormone (TR) (Wagner et al., Nature 378:690-697, 1995), retinoic acid (RAR and RXR) (Renaud et al., Nature 378:681-689, 1995; and Bourget et al., Nature 375: 377-382, 1995; respectively), estrogen (ER) (Brzozowski et al., Nature 389: 753-758, 1997), progesterone (PR) (Williams and Sigler, Nature 393: 392-396, 1998), and peroxisome proliferators (PPAR) (Xu et al., Mol. Cell 3: 397-403, 1999). Alignments comparing helix 1 sequences are described by Wurtz et al. (Nature Struct. Biol. 3: 87-94, 1996).

By “helix 12 domain” is meant that domain of a nuclear receptor that corresponds to the twelfth conserved helix of the nuclear hormone receptor ligand binding domain as defined by x-ray crystal structures, for example, those of the receptors for thyroid hormone (TR) (Wagner et al., Nature 378:690-697, 1995), retinoic acid (RAR and RXR) (Renaud et al., Nature 378:681-689, 1995; and Bourget et al., Nature 375: 377-382, 1995; respectively), estrogen (ER) (Brzozowski et al., Nature 389: 753-758, 1997), progesterone (PR) (Williams and Sigler, Nature 393: 392-396, 1998), and peroxisome proliferators (PPAR) (Xu et al., Mol. Cell 3: 397-403, 1999). This helix contains a conserved hydrophobic motif necessary for ligand-dependent activation of transcription. Alignments comparing helix 12 sequences are described by Wurtz et al. (Nature Struct. Biol. 3: 87-94, 1996).

By “ligand binding pocket” is meant that portion of a nuclear receptor that contains the amino acid moieties constituting ligand binding determinants. Such determinants may be identified by mutations that disrupt ligand binding and/or by analysis of amino acids that lie in the proximity of ligand in the x-ray crystal structures of ligand binding domains, such as those of the receptors for thyroid hormone (TR), retinoic acid (RAR and RXR), estrogen (ER), progesterone (PR), amd peroxisome proliferators (PPAR).

By “fragment” is meant a portion of a protein that is less than the full-length amino acid sequence. Typically, at least one assay fragment is between 10 and 200 amino acids, preferably, between 10 and 100 amino acids, more preferably, between 15 and 75 amino acids, and, most preferably, between about 15 and 50 amino acids in length, particularly for a helix 1 fragment. The second assay fragment may be any length and may encompass the remainder of an effector binding domain. For nuclear receptors, this fragment is typically less than 300 amino acids, preferably, less than 250 amino acids, and, more preferably, less than 200 amino acids in length.

By a “solid support” is meant, without limitation, any column (or column material), bead, test tube, microtiter dish, solid particle (for example, agarose or sepharose), microchip (for example, silicon, silicon-glass, or gold chip), or membrane to which a complex may be bound, either directly or indirectly (for example, through other binding partner intermediates such as other antibodies or Protein A).

The present invention provides long awaited advantages over a wide variety of standard screening methods used for identifying effector molecules, such as ligands. For example, this assay is based on the effector dependent assembly of a target protein of interest. The generality of this approach means that it can be applied to a wide range of different proteins, such as nuclear hormone receptors and kinases. In the case of the nuclear receptors, the assay can be adapted to large scale screens appropriate for examining many thousands of different compounds for effector function. The assay is specific for the individual receptor, allowing the design of mixed screens in which a single compound can be simultaneously tested for effector function with more than one target protein. It also differs from other assays based on interaction of the target protein with additional proteins in that it does not require the previous identification of such additional proteins.

Accordingly, the methods of the invention provide a facile means to identify effector molecules, for example, from compound or protein libraries, and in so doing provide a route for analyzing virtually any number of compounds for protein assembly-mediating capabilities with high-volume throughput, high sensitivity, and low complexity. The methods are also relatively inexpensive to perform and enable the analysis of small quantities of compounds, including active substances found in either purified or crude extract form.

Other features and advantages of the invention will be apparent from the following description of the preferred embodiments thereof, and from the claims.

### BRIEF DESCRIPTION OF THE DRAWINGS

FIGS. 1A-1C show that the hinge of TR interacts with the remainder of the LBD in a ligand-dependent manner. FIG. 1A is a schematic representation of the different domains of TR and a detailed representation of the primary and secondary structure of the hinge region. FIG. 1B shows a mammalian two-hybrid assay of the Gal-TR hinge (204-260) with progressive deletions of TR-LBD from the N-terminus fused to a VP16 activation domain. HepG2 cells were cotransfected with the above constructs along with the G5E1B-Luc reporter containing 5 Gal4 sites in front of a minimal E4 TATA box and treated with 10⁻⁶M T3 or vehicle alone. FIG. 1C shows an in vitro interaction assay using two GST fusions of TR hinge (GST-TR204-260 and a shorter GST-TR204-235) with in vitro translated [³⁵S]-methionine-labeled TR-LBD lacking helix 1 (CMXFlag-TR236-461) (top panel) or containing helix 1 (CMXFlag-TR204-461) (bottom panel).

FIGS. 2A and 2B show that full length TR separated into two fragments can assemble in a ligand dependent manner in vitro and in vivo. FIG. 2A shows an EMSA assay of fall length TR (1-461) and TR fragments (TR1-235 and TR236-461) on a DR4 oligonucleotide. In vitro translated full length TR or the fragments were used alone or in combination, in the presence or absence of 10⁻⁶M T3. In vitro translated RXR was used in all lanes. FIG. 2B illustrates cotransfection of TR fragments (TR1-235 and FlagTR236-461) with 28T-TK-Luc reporter containing two IRO elements in HepG2 cells in the absence or presence of T3.

FIGS. 3A-3D illustrate the effect of mutations within the hinge region on the ligand-dependent association between the TR hinge and the remainder of the TR-LBD. FIG. 3A shows the primary and secondary structure of the TR hinge region with point mutations reported to abolish corepressor binding. Schematic representations of the deletion and mutation constructs of the TR hinge region are also shown. FIG. 3B illustrates a mammalian two-hybrid assay: Gal fusions of the wt and TR hinge deletions were cotransfected into HepG2 cells together with VP-TR236-461 and G5E1B-Luc reporters in the presence of T3 (right panel) or vehicle alone (left panel). FIG. 3C also illustrates a mammalian two-hybrid assay: Gal fusions of the wt and TR hinge point mutations were cotransfected into HepG2 cells together with VP-TR236-461 and G5E1B-Luc reporters in the presence of T3 (right panel) or vehicle alone (left panel). FIG. 3D shows a yeast two-hybrid assay of LexA fusion of wt and mutated TR hinge constructs with a B42 fusion of TR236-461.

FIG. 4 illustrates the generality of the helix 1-LBD interaction. Shown are mammalian two-hybrid assays of Gal fusions containing helix 1 of either TR (left panel), RAR (middle panel), or RXR (right panel) with VP16 fusions of their respective LBDs containing or lacking helix 1. HepG2 cells were cotransfected with G5E1B-Luc reporter, the appropriate combination of the expression vectors, and ligands or vehicle.

FIGS. 5A and 5B illustrate that N-CoR enhances the hinge-LBD interaction in the absence of ligand. In FIG. 5A, HepG2 cells were cotransfected with Gal4-hinge and VP16-LBD fusions of TR (left panel), RAR (middle panel), or RXR (right panel) in the presence or absence of Gal-RIP13ΔN4 construct and treated with the corresponding ligands or vehicle alone. FIG. 5B shows a GST pulldown assay of GST-TR204-260 with in vitro translated TR236-461. Wt and mutant peptides of N-CoR IDI (top and bottom panels, respectively) were included where indicated at concentrations of 10 (+) and 100 (++) μM.

FIGS. 6A and 6B show that N-CoR binding induces the appearance of a smaller protease-resistant fragment. In FIG. 6A, S³⁵-labeled in vitro translated TR204-461 was incubated with 1 mM of either wt or mutated N-CoR peptide in the absence or presence of 1 μM T3. The mixture was digested with trypsin for 7 minutes at room temperature and analyzed by SDS-PAGE. In FIG. 6B, full length TR LBD204-461 (F) or a C-terminal truncated TR LBD (Δ15C) was incubated with peptides and/or T3 and digested with trypsin as above.

FIG. 7 shows a model of the allosteric effects of ligands and corepressors on the interaction between the hinge and the LBD of nuclear hormone receptors. The model is based on the released coordinates of the human TRβLBD with coactivator peptide. The hinge region including helix 1, helix 12, and the corepressor peptide are drawn as ribbons. The rest of TR LBD is represented as surface. The blue color indicates stable positions. The red color indicates instability and uncertainty in position. Ligand is visible in the holo-LBD. The drawing was created with Swiss PDB viewer.

FIG. 8 illustrates the ability of both agonists and antagonists to stimulate the helix1-LBD interaction. Shown are mammalian two-hybrid assays of Gal fusions containing helix 1 of ER-α with VP16 fusions of the ER LBD containing or lacking helix 1. HepG2 cells were cotransfected with G5E1B-Luc reporter, the appropriate combination of the expression vectors, and a ligand or vehicle. “E2” denotes estradiol; “4HT” denotes 4-hydroxy tamoxifen; and “ICI” denotes ICI-172,780.

### DETAILED DESCRIPTION

Described below are experiments demonstrating that effector molecules may be identified on the basis of their ability to mediate protein assembly. These experiments are carried out using a variety of nuclear receptors as model systems. Specifically, these experiments show that, in the presence of the effector molecule (but not in its absence), receptor fragments are able to associate and assemble, with the effector, into a stable complex. In addition, these experiments demonstrate the specificity of the assay. Also as described below, effector ligands were only able to trigger assembly of their cognate receptors and were ineffective when contacted with other receptor fragments, including fragments from other nuclear hormone receptors.

Nuclear Receptors

The nuclear hormone receptor superfamily includes the receptors for a number of important hormones and biological regulators, such as steroids, thyroid hormone, and retinoids (Mangelsdorf et al., Cell 83:835-839, 1995). These receptors are ligand dependent transcription factors that exert their effects by binding to specific DNA sequences called response elements. The receptors generally function as transcriptional activators when occupied by their ligand and a subset are also active transcriptional repressors in the absence of ligand.

The transcriptional regulatory properties of the receptors are mediated by a number of ligand-dependent receptor interacting proteins (Shibata et al., Recent Prog Horm Res 52:141-64, 1997; Xu et al., Curr Opin Genet Dev 9:140-147, 1999). These receptor interacting proteins are classified as coactivators if they are involved in transcriptional activation and corepressors if they mediate repression. The nature of the transcriptionally active complex has recently been elucidated by the solution of the x-ray crystal structures of ligand activated receptors bound by peptides corresponding to the receptor interacting motifs of coactivators (Nolte et al., Nature 395:137-43, 1998; Shiau et al., Cell 95:927-37, 1998; Darimont et al., Genes Dev 12:3343-56, 1998). In these structures, an amphipathic coactivator helix specifically contacts a hydrophobic surface of the receptor LBD that is formed by several helices. Based on these structures as well as a variety of functional studies, it is thought that the primary role of the ligand is to direct the C-terminal receptor helix 12 to adopt a specific position relative to the rest of the LBD. This allosteric shift would complete the complementary surface on the receptor recognized by the coactivator.

The structure of the receptors in the absence of ligand and the basis for the interaction of these aporeceptors with corepressors has been much less clear. Initial mutagenesis studies have suggested that the first helix in the LBD might be the target for corepressor binding (Horlein et al., Nature 377:397-404, 1995). This helix is in a region referred to as the hinge, which was initially thought to serve as a relatively flexible connection between the DNA binding domain and the LBD. However, in the crystal structures of the thyroid hormone receptor (TR) and the retinoic acid receptor (RAR) this helix was found to be folded tightly into the LBD and the residues thought to contact corepressors were not accessible to the surface (Wagner et al., Nature 378:690-697, 1995; Bourget et al., Nature 375:377-382, 1995). More recent studies have suggested a quite different model for corepressor binding, in which the receptor surface recognized overlaps with that bound by the coactivators (Hu and Lazar, Nature 402:93-96, 1999). This model accounts well for the effects of receptor mutations in that surface on coactivator and corepressor binding, and also for similarities in the amphipathic helices of both coactivators and corepressors that specifically contact receptors. However, important aspects of the basis for the switch between the corepressor binding and coactivator binding forms remain unclear.

Ligand-Dependent Receptor Assembly

As described in more detail below, using a novel approach based on reassembling fragments of the LBD, we found that a fragment consisting of the amphipathic helix 1 of TR associates strongly and specifically with the rest of the TR LBD in the presence of hormone, as indicated by both in vitro and in vivo assays. Similar results were observed for RAR and the retinoid X receptor (RXR). In striking contrast, helix 1 of these receptors showed only a very weak interaction with the rest of the LBD in the absence of ligand. This lack of interaction was unexpected, since in the absence of ligand helix 1 is tightly associated with the rest of the LBD in the crystal structure of RXR and the peroxisome proliferator activated receptor (PPAR) (Bourget et al., Nature 375:377-382, 1995; Uppenberg et al., J Biol Chem 273:31108-12, 1998).

The structures of the receptor LBDs, like those of other globular proteins, are based on an internal hydrophobic core composed of several tightly packed helices (Wurtz et al., Nature Struct. Biol. 3:87-94, 1996). This core is continuous with the rather large hydrophobic binding surface for coactivators and corepressors, and also with the ligand binding pocket. Since binding of the hydrophobic ligand should clearly contribute to the stability of this core, the ligand dependence of the interaction of helix 1 with the remainder of the LBD is likely a reflection of this stabilization. However, the energetically unfavorable exposure of the coactivator/corepressor surface to solvent should destabilize the core, particularly in the absence of ligand. Thus, any mechanism preventing such exposure of this surface of the aporeceptor should also stabilize helix 1 interaction. This prediction was strongly supported by results demonstrating that corepressor binding also significantly stabilized the aporeceptor, as demonstrated by its ability to promote helix 1 to interact with the remainder of the LBD. Overall, these results suggested that ligand binding not only promoted correct formation of the coactivator binding surface, but also had a much more profound and dynamic effect on the overall structure of the LBD.

Ligand-Dependent Association of the Hinge Region With the Ligand Binding Domain of TR

The hinge region of the nuclear receptors was defined a number of years ago on the basis of a lack of sequence conservation relative to the DNA binding domain and the ligand binding domain (LBD) (Krust et al., Embo J 5:891-7, 1986; Gronemeyer et al., Embo J 6:3985-94, 1987). Functional studies suggested that it acted simply as a relatively flexible connector between these domains (Green and Chambon, Nature 325:75-78, 1987; Thompson and Evans, Proc. Natl. Acad. Sci. USA 86: 3494-3498, 1989). However, a more direct role in transcriptional repression was also suggested by results indicating that specific mutations in this region led to a loss of corepressor binding (Horlein et al., Nature 377:397-404, 1995).

The reported x-ray crystal structure of the TR and RAR revealed that at least the C-terminal portion of the previously defined hinge region is actually incorporated into the conserved structure of the LBD itself, forming the helices 1 and 2 (Wagner et al., Nature 378:690-697, 1995; and Renaud et al., Nature 378:681-689, 1995). These structural studies raised questions regarding the postulated role of this region in transcriptional repression, because the residues proposed to interact specifically with corepressors are not accessible to the receptor surface. Instead, the proposed corepressor targets lie within the amphipathic helix 1 and project into a hydrophobic core.

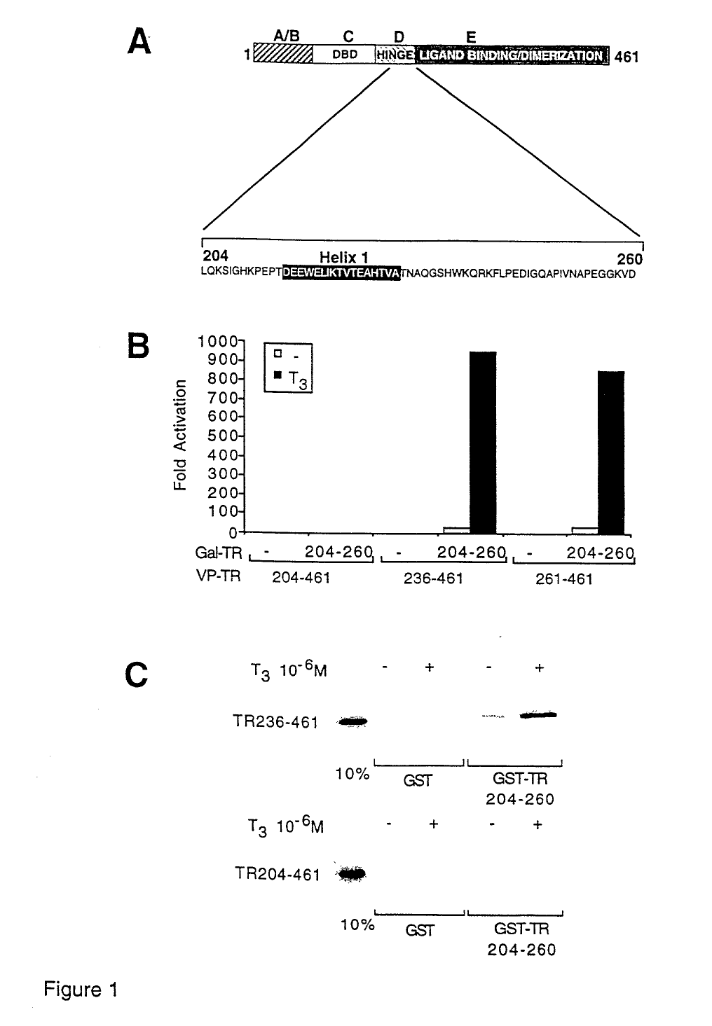

To explore the role of this region in receptor structure and function, we examined its functional interaction with the remainder of the LBD. Initial studies employed the mammalian two-hybrid system. In one hybrid, the hinge region (residues 204-260) of the human TRβ was fused to the Gal4 DBD. This segment extended from the C-terminus of the extended DNA binding domain through helices 1 and 2 of the TR-LBD, terminating before the beginning of helix 3 (FIG. 1A). In the other hybrid, progressive N-terminal deletions of the TR-LBD affecting this region were fused to the VP16 activation domain.

This approach demonstrated a strong interaction of the hinge with the rest of the LBD in the presence of T3. Quite unexpectedly however, only a modest residual interaction was observed in the absence of hormone (FIG. 1B). This lack of interaction was surprising, since currently available LBD structures show helix 1 of the hinge occupying essentially the same position in both the apo-and in the holo-LBD structures (Bourget et al.., Nature 375:377-382, 1995; and Uppenberg et al., J. Biol. Chem. 273(47):31108-31112, 1998). Thus, the relationship of this region to the rest of the LBD was not expected to be altered by the presence or absence of ligand.

This hormone-dependent interaction was only observed when helix 1 was missing from the LBD fragment. This indicated that the interaction of the helix 1 region with the remainder of the LBD in this system was not based on some secondary site of interaction elsewhere on the LBD, and was likely to reflect the normal association of the two pieces in the intact LBD structure.

To determine whether a similar hormone-dependent interaction could be observed biochemically, the TR hinge region was fused to GST and tested for association with appropriate TR LBD fragments. The ligand dependence of the TR hinge-LBD interaction observed strongly confirmed the in vivo results from the mammalian two-hybrid assay (FIG. 1C).

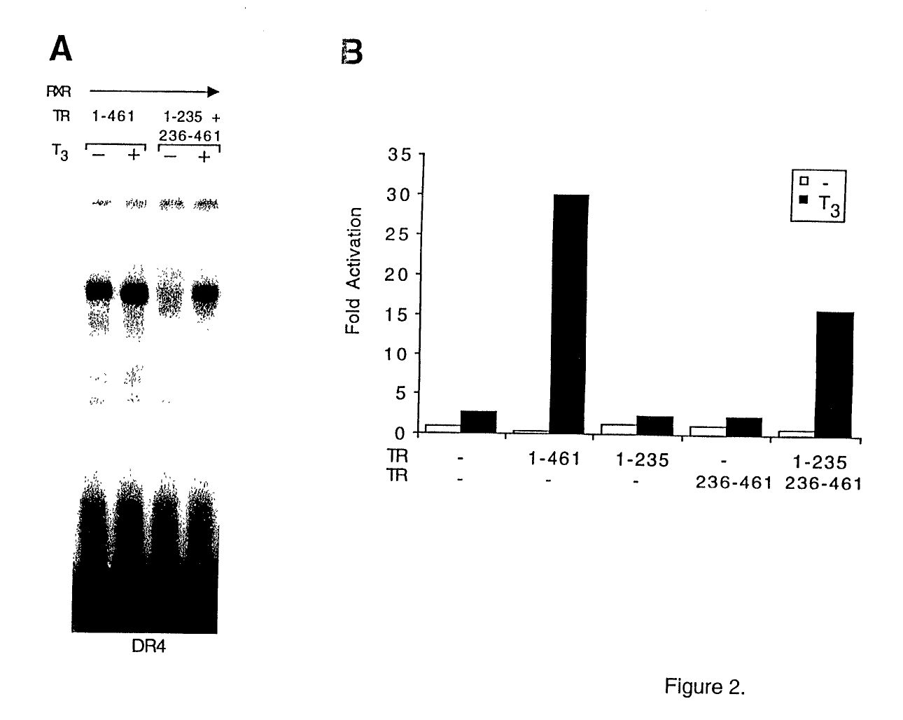

To further confirm the ligand-dependent association between the hinge and the LBD, their interaction was examined in the context of the full length TR. Thus, a fragment of TR including the N-terminus, DNA binding domain, and hinge through helix 1 was tested for interaction with a fragment consisting of the remainder of the TR LBD. For electrophoretic mobility shift assays (EMSA), these fragments were separately translated in vitro and incubated, either singly or in combination, with RXR and a TR/RXR heterodimer DNA binding site. As expected from the previous results, the two TR fragments were only able to reconstitute a specific DNA binding complex in the presence of T3. The ligand-dependent interaction of the two TR fragments was also confirmed in vivo by cotransfections in which the two pieces were expressed from separate mammalian expression vectors, along with an appropriate T3 responsive reporter. The transactivation of the reporter observed in the presence of ligand effectively demonstrated the assembly of the two TR fragments to generate a functional receptor (FIG. 2B).

Mutations in the Hinge Affect the Interaction With the Ligand Binding Domain

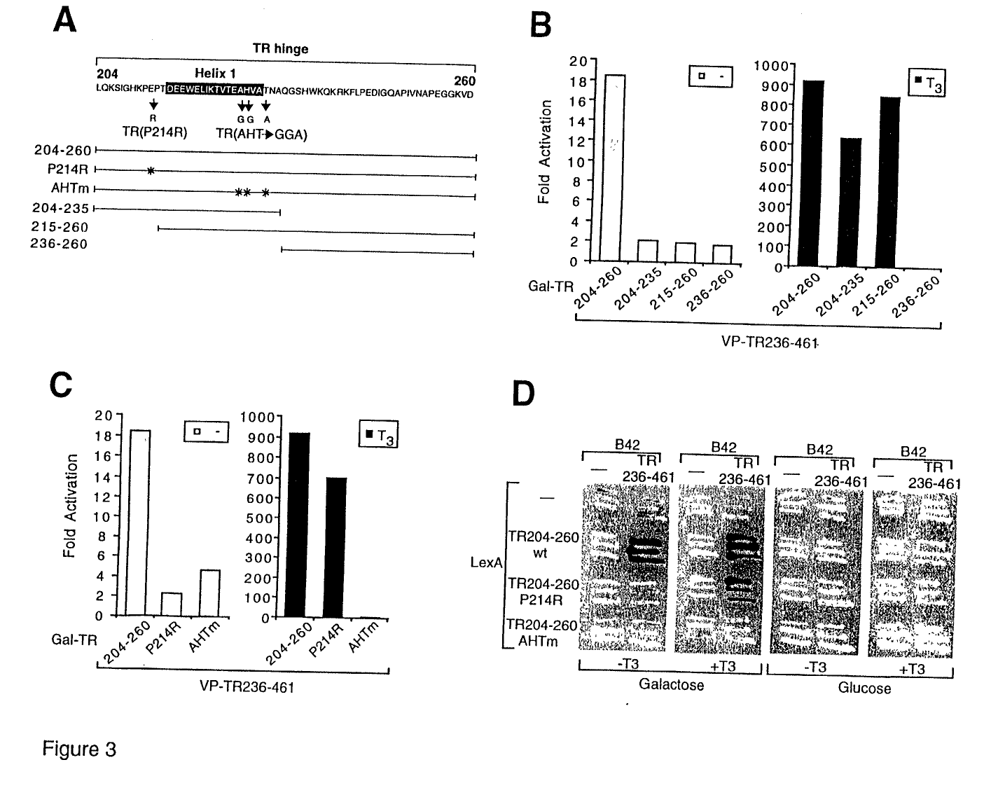

The interaction of the hinge region of TR (204-260) with the remainder of the LBD was characterized in more detail using a series of deletions and point mutations (FIG. 3A). The effect of these mutations on the interaction in the presence or absence of T3 was evaluated using the mammalian two-hybrid assay. In the presence of ligand, helix 1 was found to be necessary for ligand-dependent association between the hinge and the LBD (FIG. 3B). Neither helix 2 and more C-terminal residues nor residues N-terminal to helix 1 was required. However, loss of either helix 2 and C-terminal sequences or the N-terminal sequences did preclude the minimal interaction observed in the absence of ligand. A triple point mutant previously described as blocking corepressor interaction (AHT-GGA) (Horlein et al., Nature 377:397-404, 1995) completely disrupted interaction of helix 1 with the LBD not only in the absence of hormone, but also in its presence (FIG. 3C). This suggested that these mutations affected the overall stability of the LBD, which is in accord with the fact that the mutated residues contribute to the hydrophobic core of the receptor, and also the observation that this triple mutation blocks heterodimerization with RXR. In contrast, the P214R mutant, which was also reported to block corepressor function (Damm and Evans, Proc Natl Acad Sci USA 90:10668-72, 1993), specifically blocked the residual interaction observed in the absence of ligand (FIG. 3C).

The effects of the point mutations on the TR hinge-LBD interaction were confirmed in the sensitive yeast two-hybrid assay (FIG. 3D). In this context, the residual interaction between the hinge and the remainder of the LBD was more evident. As expected from the mammalian two hybrid results, the triple mutant blocked both the basal and ligand induced interaction, while the P214R mutation blocked only the basal interaction.

Overall, we concluded that the association between the hinge region and the remainder of the TR LBD was driven by the amphipathic helix 1. The observation that point mutations within this helix block its ability to interact with the apo-TR LBD suggests that the effect of these mutants on TR function in the absence of ligand is due to an indirect effect on stability, rather than a direct effect on corepressor interaction.

Generality and Specificity of Helix 1 LBD Interaction

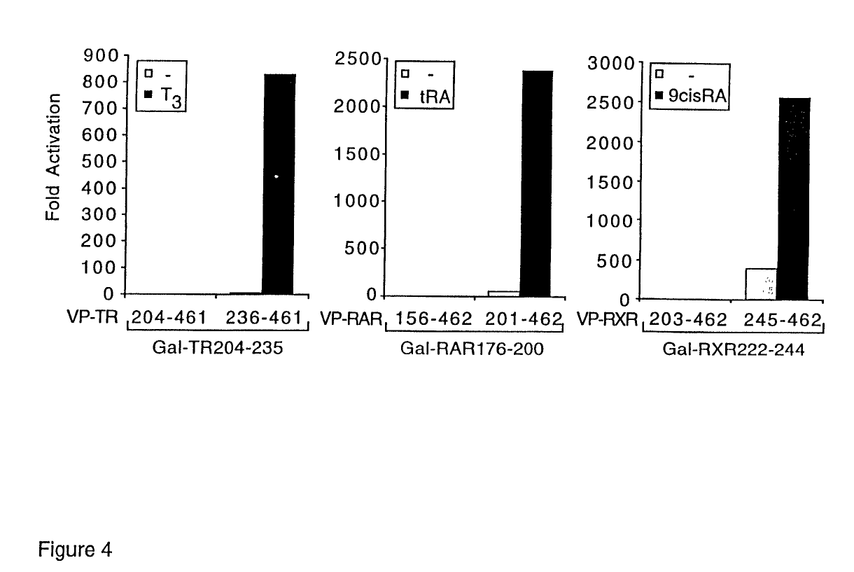

The generality of the ligand-dependent association between helix 1 and the remainder of the LBD was tested using RAR and RXR. For both of these receptors, strong interaction was observed in the presence of ligand (FIG. 4). In the absence of ligand, helix 1 of RAR showed quite limited interaction, comparable to that observed with TR. In contrast, significantly stronger interaction was observed with apo-RXR (FIG. 4).

To test the specificity of the observed helix 1-LBD interactions, all possible pair-wise combinations of helix 1 and LBD fragments were tested in the mammalian two hybrid assay. As expected from the significant divergence of the helix 1 sequences, effective interaction was only observed with the cognate pairs.

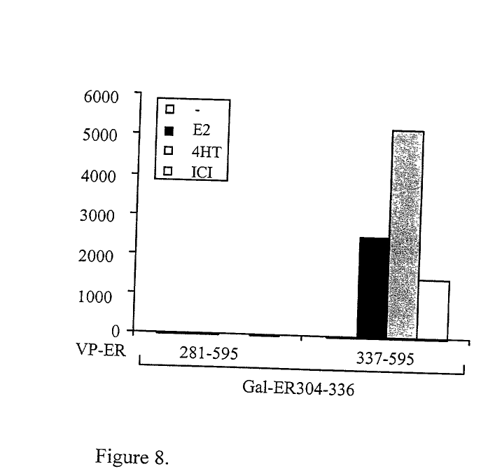

In addition to testing the ability of ligands to enhance the hinge-LBD interaction for the retinoid receptors RAR and RXR, we demonstrated that the estradiol agonist stimulates the hinge-LBD interaction of the steroid receptor ER-α (FIG. 8). Similar to the results for apo-TR and apo-RAR, limited interaction was observed between helix 1 and the LBD of ER in the absence of ligand.

Antagonists Enhance the Hinge-LBD Interaction

Because both agonists and antagonists can confer protease resistance in ER and other steroid receptors, we hypothesized that antagonist binding may also stabilize the hinge-LBD interaction (Allan et al., J. Biol. Chem. 267:19513-19520; McDonnell et al., Mol. Endocrinol. 9:659-669, 1995). This hypothesis was tested by determining the effect of antagonists on the hinge-LBD interaction of ER-α. The ER antagonist ICI-172,780 significantly enhanced this interaction (FIG. 8). A stronger effect was induced by 4-hydroxy tamoxifen, which can function as an ER partial agonist or as an antagonist depending on the particular cell type and promoter being assayed (FIG. 8) (McDonnell et al., Ann. NY Acad. Sci. 761:121-137, 1995; McDonnell et al., Mol. Endocrinol. 9:659-669, 1995). In particular, the partial agonist activity of 4-hydroxy tamoxifen is thought to depend on the amino-terminal transactivation TAF-1 domain of ER, which is not present in the ligand binding domain fragments used in the mammalian two-hybrid assay described herein. Thus, in the mammalian two-hybrid assay, 4-hydroxy tamoxifen may function as an antagonist.

These results demonstrate that both agonists and antagonists enhance the hinge-LBD interaction of ER-α.

Corepressors Enhance the Hinge-LBD Interaction

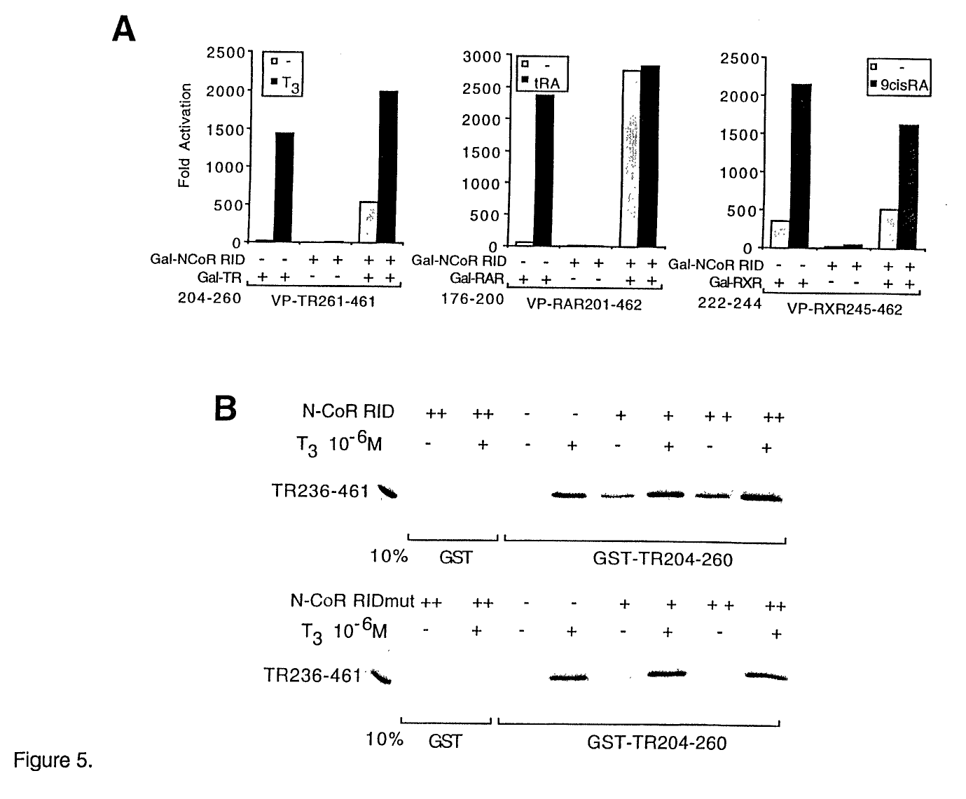

We hypothesized that corepressor binding might also stabilize the aporeceptor structure, since such binding could counteract the destabilizing effect of solvent exposure of the coactivator/corepressor binding surface (Leng et al., J. Steroid Biochem. Mol. Biol. 46:643-661, 1993). This hypothesis was initially tested by examining the effects of a Gal4-NCoR fusion on the hinge-LBD interaction in the mammalian two-hybrid assay. This NCoR protein, which included both receptor interaction domains, strongly stimulated the hinge-TR and hinge RAR interaction in the absence of ligand (FIG. 5A). As expected, it had no effect in the presence of ligand, and also did not significantly affect the hinge-RXR interaction in either the presence or absence of ligand.

The effect of corepressor binding on the hinge-LBD interaction was tested more directly biochemically. As expected, increasing amounts of a peptide corresponding to the minimal NCoR receptor interaction domain (RID) I strongly stimulated the interaction between a GST-TR hinge fusion and the TR LBD in the absence of ligand (FIG. 5B). This effect was quite specific, as the peptide did not affect interaction in the presence of ligand, and a mutant NCoR peptide unable to interact with receptors was without effect in either the presence or absence of ligand.

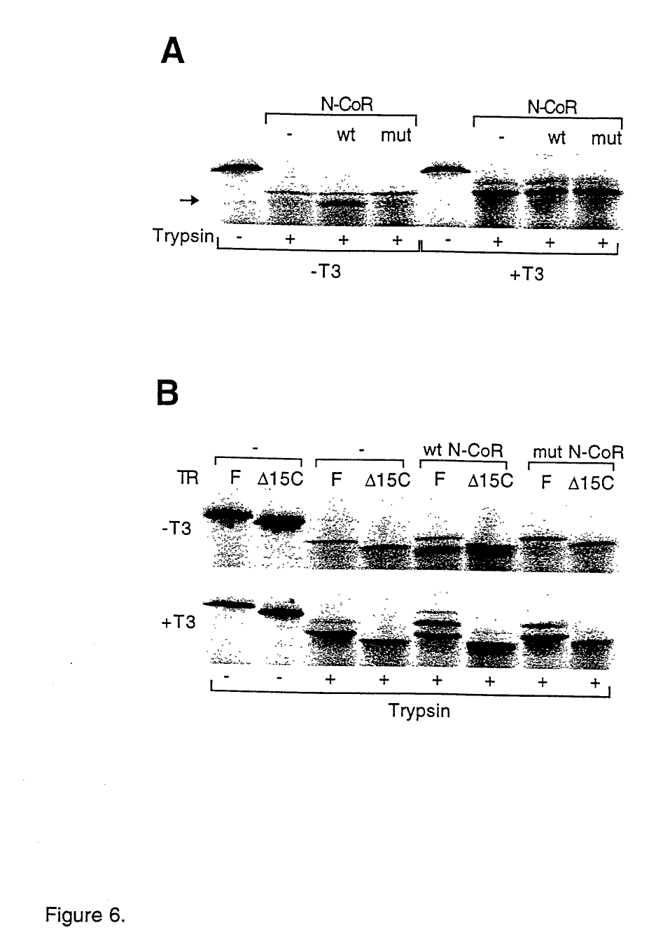

To further test the consequences of corepressor binding to the TR LBD, we examined the effect of the peptide on protease cleavage of a fragment containing this domain and some additional N-terminal sequences. The hormone bound LBD was relatively resistant to degradation by trypsin. In the absence of ligand, a fragment corresponding to this domain could be observed at lower levels of digestion, but this species was lost when either the time of incubation or the amount of protease were increased. Addition of the wild type NCoR RID I peptide to such digestions in the absence of ligand resulted in both some apparent stabilization and also the presence of a new protease resistant species with a lower molecular weight (FIG. 6A). This species was not observed in the presence of the mutant peptide and, as expected, neither peptide had any effect on cleavage in the presence of ligand. It is important to note that, while the stabilization could be due to steric inhibition of a trypsin cleavage site, this cannot account for the production of a smaller protected product. Thus, the presence of the new protected species strongly confirmed that corepressor binding specifically altered TR LBD structure.

The small shift in molecular weight of the protected fragment generated by the NCoR peptide could be due to cleavage at either the N- or C-termini of the LBD. A priori, the former seems inconsistent with the stabilizing effect of this peptide on helix 1. However, the latter would be consistent with corepressor binding to the coactivator site, since this should prevent the C-terminal helix 12 from binding this surface and increase its sensitivity to protease cleavage. To determine whether the smaller species was produced by N- or C-terminal cleavage, a TR LBD fragment lacking 15 C-terminal residues was examined. This deletion was chosen to remove the predicted C-terminal tryptic product. As expected, the size of the resistant species observed on digestion of this C-terminally truncated TR LBD in the presence of the wild type NCoR RID I peptide was very similar to that of the novel product observed with the wild type TR LBD (FIG. 6B). Importantly, the protected fragment observed with this truncated LBD in the presence of hormone was shorter than the wild type fragment by the expected amount. This confirmed that the C-terminus of the wild type TR was protease resistant in the presence of ligand. Addition of sequences to the N-terminus did not affect the size of the protected species, indicating that the initial cleavage products were missing the N-terminus (data not shown). Overall, we concluded that corepressor binding to the TR LBD generated a novel protease resistant fragment that was missing the C-terminus of the protein.

Effector-Mediated Nucleic Receptor Structural Changes

Ligand binding to a nuclear hormone receptor results in the formation of a specific binding surface for coactivators. Both functional and structural studies demonstrate that this surface consists of a hydrophobic groove, surrounded by helices 3, 5, and 12. Analysis of an increasing number of x-ray crystal structures has suggested that ligand binding exerts a specific allosteric effect on the position of the C-terminal helix 12, which contains a conserved motif required for ligand dependent transactivation. In contrast to the mobility of helix 12, much of the remainder of the LBD is thought to be maintained in an invariant, canonical structure that is based on a hydrophobic core including several tightly packed helices (helices 1, 5, 8, 9, and 10 in TR) (Wurtz et al., Nature Struct. Biol. 3:87-94, 1996; Moras and Gronemeyer, Curr Opin Cell Biol 10:384-91, 1998; Blondel et al., J. Mol Biol 291:101-15, 1999). The relative stability of this core region was recently supported by its lack of mobility in a recent detailed molecular dynamics based simulation of the exit of ligand from RARβ.

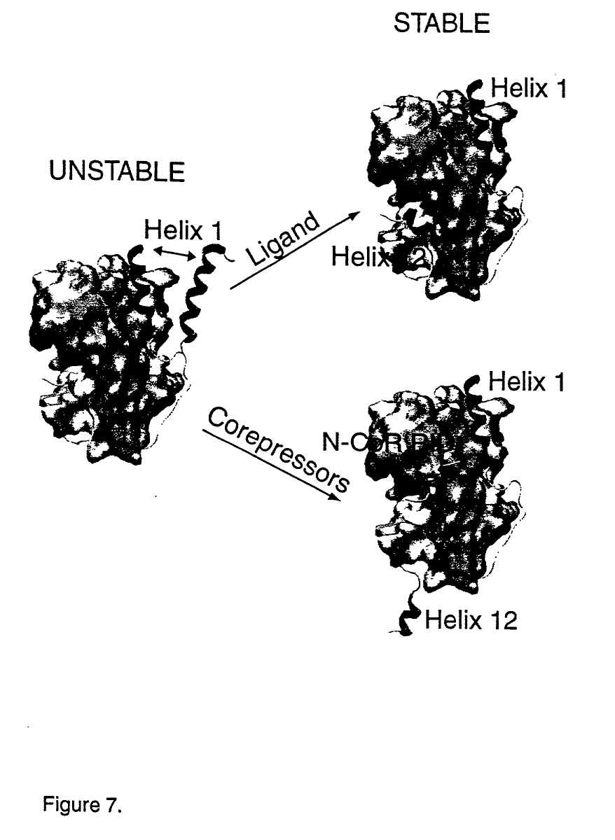

The results described here are consistent with a more dynamic picture. In this model (FIG. 7), the structure of the receptor in the absence of ligand is presumed to be generally similar to that of the hormone bound form. However, the aporeceptor is unstable relative to the hormone bound structure, even in the hydrophobic core. This lack of structural integrity of the LBD results in the specific loss of the ability of a helix 1 fragment of either TR or RAR to stably associate with the rest of the LBD in the absence of ligand. Interestingly, an intermediate level of interaction is observed between helix 1 of RXR and the remainder of the RXR LBD in the absence of ligand. This is quite consistent with the recent demonstration that apo-RXR is more stable than apo-TR. Thus, it is likely that ligand binding not only generates the proper coactivator interface by appropriately positioning helix 12, but also significantly and globally stabilizes receptor structure. Importantly, the results with helix 1 indicated that this stabilization effect is evident even in regions of the hydrophobic core that do not contact the ligand directly.

Binding of both coactivators and corepressors to a particularly hydrophobic surface on receptor LBDs suggests a specific explanation for the relative instability of the aporeceptor LBD. In the absence of ligand, this relatively large surface is apparently exposed to solvent. Such exposure should be quite destabilizing, particularly as this surface is continuous with the hydrophobic core thought to define the overall LBD structure. In the presence of ligand, helix 12 partially covers this surface, which may account for at least a portion of the stabilizing effect of ligand. In the case of the aporeceptor, however, the hydrophobic surface can also be covered by binding of corepressor. This leads to the novel prediction that corepressor binding should also directly stabilize the LBD structure, which is supported by the results described here.

These results also support an additional, more specific prediction diagrammed in FIG. 7. In modeling studies, binding of corepressor to the coactivator/corepressor site precludes helix 12 from adopting a position analogous to that of the ligand activated state. It should also prevent the amphipathic helix 12 itself from occupying the coactivator site, as recently described for the antagonist bound state of the estrogen receptor and a particular crystal form of the apo-PPAR. This displacement of helix 12 from the coactivator/corepressor site would be expected to increase its sensitivity to proteolytic cleavage. The demonstration that corepressor binding results in a new proteolytically stabilized fragment lacking helix 12 strongly supports this prediction.

Materials and Methods

The above experiments were carried out using the following materials and methods.

Plasmid construction

Gal4 and VP16 fusions of the various deletions of TR, RAR, RXR, and ER-α were constructed by amplification of the corresponding region and subcloned as SalI/NotI fragments into modified CMX-Gal4 and CMX-VP16 vectors. The products were either extensively sequenced or most of the amplified region was removed by restriction digestion and replaced with an unamplified fragment. Point mutations within the hinge region of TR were introduced by two rounds of amplification. First, two overlapping oligonucleotides bearing the mutations were used to create mutated templates. In the second round external primers and the mutated templates were used to incorporate the mutations in the entire hinge region. The appropriate mutated constructs were transferred by restriction digestion into GST, LexA, or B42 vectors.

Cell culture and transfections

HepG2 cells were maintained in Dulbecco's modified Eagle medium supplemented with 10% fetal calf serum. For transfection the cells were plated the day before into 24-well dishes at density 10⁵ cells/well in DMEM supplemented with 10% charcoal-stripped serum. Transfections were performed by the calcium phosphate co-precipitation method. Typically 200 ng of reporter plasmid, 50 ng of β-Galactosidase expression vector as an internal control, and 2-50 ng of expression vectors were used. The amount of DNA was adjusted to 500 ng total per well with pBluescript vector.

The cells were washed with PBS the next morning and fresh media containing the appropriate ligands was added. The cells were harvested 24 hours later and assayed for luciferase (Molecular Probes) and β-Galactosidase activity (Tropix) on an MLX luminometer. All experiments were performed at least twice in duplicate.

GST pulldown assays

The appropriate GST fusion constructs were induced with 0.2 mM in BL21 cells and coupled to glutathione beads (Sepharose, Pharmacia). Interactions were performed overnight at 4° C. in buffer containing 20 mM Hepes pH 7.6, 100 mM NaCl, 20% glycerol, 2 mg/ml BSA, 1 mM DTT, and various protease inhibitors. The beads were subsequently washed twice with the interaction buffer containing 0.05% Triton X-100 and once with 50 mM Tris-HCl pH 8. Ligands or peptides were included in the washes where appropriate. The bound proteins were eluted with 15 mM glutathione (Sigma) for 15 minutes at 37° C. and analyzed by SDS-PAGE and fluorography (Amplify, Amersham). The sequence of the N-CoR RID peptide was ASNLGEDIIKALMGSFDD.

Electrophoretic mobility shift assay (EMSA)

pT7HisMyc-hRXRa, CMX-TR(F), CMX-TR1-235, and CMX-FlagTR236-461 were translated in vitro using the TNT T7 coupled transcription-translation system (Promega). DR4 oligonucleotide was end-labeled with γ-³²-P-ATP (NEN) using T4 polynucleotide kinase (New England Biolabs). 1 ml of the in vitro translated proteins and the probe were incubated in a buffer containing 20 mM Hepes pH 7.6, 50 mM KCl, 1 mM DTT, 5 mM MgCl₂, 5% Ficoll, and 100 ng polydI/dC:polydI/dC in the presence of 10⁻⁶ M T3 or vehicle. After 30 minutes at room temperature, the mixture was analyzed on 4% polyacrylamide gels in 0.5× TBE buffer. Gel were subsequently dried and developed by autoradiography.

Yeast two-hybrid

The yeast two hybrid assay was performed in EGY48 strain essentially as described (Ausubel et al., Current Protocols in Molecular Biology, 1999).

Protease Digestion Assay

For the protease digestion assay, 2.5 ml of ³⁵-S-methionine-labeled TR 204-461 and TR 204-446 (D15C) were incubated with either ETOH or T3 (1 mM final) for 15 minutes on ice. Subsequently, wild-type or mutated N-CoR peptides were added to final concentrations of 1 mM, and the reaction mixtures was further incubated for 30 minutes on ice. BSA was added to the reactions with no peptides to supplement for equal protein concentration in all the reaction mixtures. Finally, 0.5 mg of trypsin was added to the indicated tubes, and the protein digestion was performed at room temperature for 7 minutes. The reactions were stopped with the addition of SDS-sample buffer and boiling. The samples were run on a 15% SDS-gel that was then dried and exposed to film for 1 hour.

Effector-Dependent Assembly Assays

The effector-dependent assembly of protein fragments, as exemplified by the ligand dependent assembly of receptor LBD fragments described herein, provides the basis for the development of unique assays for effector identification. Specifically, candidate effectors may be screened using protein fragments in order to identify molecules capable of mediating protein fragment assembly. Any number of assay formats may be utilized to carry out these screens.

In one preferred example for carrying out rapid, large scale assays, a first protein fragment is immobilized on a solid support by any technique and contacted with the second, labeled protein fragment in the presence of candidate effector molecules. Specific binding of an effector is signaled by the association of label with the solid support, indicating that the effector has mediated protein fragment assembly. The protein fragments may be generated by any synthetic, recombinant, or amplification technique and may be labeled by any standard labeling approach. The solid support may include, for example, any plate, chip, bead, or column, and the protein fragment may be directly bound to the support or may be attached indirectly, for example, through interactions mediated, for example, by binding pairs such as GST/glutathione, antibody/antigen, or avidin/biotin moieties. Complexes may be identified simply by detecting label (for example, radioactive, fluorescent, chromogenic, or chemiluminescent label) associated with the solid support, either by direct visualization or indirectly (for example, by detecting a labeled antibody specific for, and bound to, the non-immobilized protein fragment). Following identification of complexes by way of solid support-associated label, the complex is disrupted, and the effector molecule may be obtained. One exemplary technique for carrying out this general type of assay is described, for example, in Krey et al., Mol. Endocrinol. 11(6):779-791, 1997.

Alternatively, any other biochemical assay may be utilized to detect effector-mediated assembly. For example, for DNA binding proteins, an EMSA assay (for example, as described herein) may be used to identify effector molecules. In this assay, protein fragments (for example, in vitro translated fragments) are combined with nucleic acid that includes the protein's binding site, and the ability to bind to the DNA site in the presence of effector molecule is assayed. As above, protein fragments may be generated by any technique, but must include domains required for both effector and DNA binding. The DNA binding site may be any sequence capable of recognition by the protein. In this assay, either one or both of the protein components or the DNA fragment may be detectably labeled.

In yet another effector identification approach, any two-hybrid or interaction trap technique may be utilized. In this approach, one of the protein fragments is fused to a DNA binding domain and the other fragment is fused to a gene activation domain, and effectors are identified by their ability to facilitate fragment-dependent protein assembly and accompanying reporter gene expression. Any two hybrid or interaction trap technique may be used, and these techniques may be carried out in vivo (for example, in yeast or mammalian cells) or in vitro.

In addition, techniques capable of detecting assembly of the protein fragments in solution may be employed. These include any technique such as centrifugation or light scattering able to detect changes in apparent molecular weight of the assembled complex. Alternatively, spectroscopic techniques to detect structural changes may be used.

The fragments utilized in these assays may be derived from any portion of a target protein that is sensitive to the effector. In the case of the nuclear hormone receptors, this region encompasses the ligand binding domain, which includes sequences extending from the conserved helix 1 through helix 12 in x-ray crystal structures, for example, those of the receptors for thyroid hormone (TR), retinoic acid (RAR and RXR), estrogen (ER), progesterone (PR), and peroxisome proliferators (PPAR), as described above. This domain is operationally defined as consisting of the minimal sequences required for ligand binding, and in some cases may be shorter than the total region from helix 1 to helix 12, for example, for the thyroid hormone receptor, which is able to bind ligand in the absence of helix 12. In the preferred choice of fragments for nuclear hormone receptors, one fragment corresponds to helix 1 and the other corresponds to the remainder of the ligand binding domain, as described above. In other cases, two or more fragments able to reconstitute a functional ligand binding domain may be used. Such fragments may represent any of a large number of potential combinations of individual helices from the ligand binding domain, for example, fragment combinations consisting of helices 1-2 and 3-12, or helices 1-3 and 4-12, or helices 1-3, 4-9, and 10-12. Moreover, using known receptor-effector systems, any combination of fragments may be tested, for example, using the assays described herein, for their ability to assemble in the presence of the known effector. These fragment combinations may then be utilized in assays for identifying new, unknown effectors.

The assays described herein can also be adapted to proteins that are not nuclear receptors. For example, one fragment may correspond to a sequence flanking the conserved catalytic core of a protein kinase. In the case of protein kinase A (PKA), the first fragment is derived from the amino terminal region spanning amino acids 1 to 39 that includes the helix A domain (amino acids 10-31) and the second fragment is derived from the remainder of the protein. In this assay, the kinase substrate stabilizes the protein (and particularly, the PKA catalytic core) (Herberg et al., Protein Sci. 6: 569-579, 1997), promoting assembly of the protein fragments.

The choice of effector candidates can include any type of molecule, including, without limitation, proteins and organic compounds, and these effectors may be included in large populations. If desired, assays may be carried out with pools of candidates which are then further evaluated and condensed to a few active and selective materials.

Effectors may be identified from libraries of natural product or synthetic (or semi-synthetic) extracts or chemical libraries according to methods known in the art. Those skilled in the field of drug discovery and development will understand that the precise source of test extracts or compounds is not critical to the effector screening procedure(s) of the invention. Accordingly, virtually any number of chemical extracts or compounds can be screened using the methods described herein. Examples of such extracts or compounds include, but are not limited to, animal-, plant-, fungal-, or prokaryotic-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). Alternatively, libraries of natural compounds in the form of animal, bacterial, fungal, and plant extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries may be produced, if desired, according to methods known in the art, e.g., by standard extraction and fractionation methods or recombinant DNA techniques. Furthermore, if desired, any library or compound is readily modified using standard chemical, physical, or biochemical methods.

Those skilled in the art of drug discovery and development readily understand that methods for dereplication (e.g., taxonomic dereplication, biological dereplication, and chemical dereplication, or any combination thereof) or the elimination of replicates or repeats of materials already known for their effector (for example, ligand) activity should be employed whenever possible.

When a crude extract is found to have effector activity, further fractionation of the positive lead extract may be necessary to isolate chemical constituents responsible for the observed effect. Thus, the goal of the extraction, fractionation, and purification process is the careful characterization and identification of a chemical entity within the crude extract having effector activity. Methods of fractionation and purification of such heterogenous extracts are known in the art. If desired, effectors shown to mediate protein assembly may also be chemically modified according to methods known in the art.

### Other Embodiments

While the examples described above focus on ligand-mediated receptor assembly, similar assembly assays may be used to detect substrate binding or other structural changes in other proteins (for example, kinases). These assays may be carried out using any of the approaches described herein.

All publications and patent applications mentioned in this specification are herein incorporated by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the appended claims.

Other embodiments are within the claims.

What is claimed is:

1. A method for identifying an effector molecule which modulates the activity of a protein, said method comprising:
(a) providing at least two fragments of said protein;
(b) contacting said fragments with a candidate molecule; and
(c) assaying for assembly of said fragments, whereby a level of fragment assembly in the presence of said candidate molecule that is greater than the level in its absence identifies said candidate as an effector molecule which modulates the activity of said protein.

2. The method of claim 1, wherein said protein is a member of the nuclear receptor superfamily.

3. The method of claim 2, wherein said nuclear hormone receptor is the thyroid hormone receptor, retinoic acid receptor, retinoid X receptor, or estrogen receptor.

4. The method of claim 1, wherein said effector molecule is a protein.

5. The method of claim 1, wherein said effector molecule is a ligand.

6. The method of claim 1, wherein said effector molecule is a corepressor.

7. The method of claim 1, wherein said effector molecule is an antagonist.

8. The method of claim 2, wherein one of said fragments comprises the helix 1 domain of said nuclear receptor.

9. The method of claim 8, wherein the second of said fragments comprises the helix 12 domain and ligand binding pocket of said nuclear receptor.

10. The method of claim 1, wherein one or both of said fragments is provided by expressing a fragment-encoding nucleic acid.

11. The method of claim 1, wherein said contacting step (b) occurs in vivo.

12. The method of claim 1, wherein said contacting step (b) occurs in vitro.

13. The method of claim 1, wherein one of said fragments is immobilized on a solid support.

14. The method of claim 13, wherein the second of said fragments is detectably labeled.

15. The method of claim 14, wherein said assaying step (c) comprises detecting said label in association with said solid support.

16. The method of claim 1, wherein said protein is a DNA binding protein.

17. The method of claim 16, wherein said contacting step (b) occurs in the presence of a nucleic acid that comprises the binding site for said DNA binding protein and said assaying step (c) comprises detecting binding of said assembled fragments to said binding site.

18. The method of claim 1, wherein one of said fragments is covalently bound to a DNA binding domain and the other said fragment is covalently bound to a gene activation domain.

19. The method of claim 18, wherein at least one of said DNA binding domain and said gene activation domain is a heterologous domain.

20. The method of claim 18, wherein said contacting step (b) is carried out in the presence of a reporter gene operably linked to a binding site for said DNA binding domain.

21. The method of claim 20, wherein said assaying step (c) comprises detecting expression of said reporter gene.

22. The method of claim 21, wherein said assaying is carried out in a yeast or mammalian cell.

23. A kit for identifying a nuclear receptor effector molecule, said kit comprising:
(a) a first fragment of said nuclear receptor, said first fragment comprising the helix 1 domain of said nuclear receptor; and
(b) a second fragment of said nuclear receptor, said second fragment comprising the helix 12 domain and the ligand binding pocket of said nuclear receptor.

24. The kit of claim 23, wherein said protein is a member of the nuclear receptor superfamily.

25. The kit of claim 24, wherein said nuclear hormone receptor is the thyroid hormone receptor, retinoic acid receptor, retinoid X receptor, or estrogen receptor.

26. The kit of claim 23, wherein said effector molecule is a ligand or corepressor.

27. The kit of claim 23, wherein said effector molecule is an antagonist.

28. The kit of claim 23, wherein at least one of said fragments is detectably labeled.

29. The kit of claim 23, wherein one of said fragments is immobilized on a solid support.

30. The kit of claim 23, wherein one of said fragments is covalently bound to a DNA binding domain and the other said fragment is covalently bound to a gene activation domain.

31. The kit of claim 30, wherein said kit further comprises a nucleic acid comprising a binding site for said DNA binding domain.
